(19) United States
(12) Patent Application Publication
(10) Pub. No.: US 2017/0105647 A1
(43) Pub. Date: Apr. 20, 2017

(54) SYSTEMS AND METHODS FOR IDENTIFYING NEUROBIOLOGICAL BIOMARKERS USING EEG

(71) Applicant: Children's Medical Center Corporation, Boston, MA (US)

(72) Inventor: Frank Hopkins DUFFY, Boston, MA (US)

(73) Assignee: Children's Medical Center Corporation, Boston, MA (US)

(21) Appl. No.: 15/311,313

(22) PCT Filed: May 15, 2015
(86) PCT No.: PCT/US2015/031060

§ 371 Date: Nov. 15, 2016

Related U.S. Application Data
(60) Provisional application No. 61/993,623, filed on May 15, 2014.

(51) Int. Cl. A61B 5/0484; A61B 5/00; A61B 5/16; A61B 5/048

(52) U.S. Cl. CPC A61B 5/04845; A61B 5/048; A61B 5/4803; A61B 5/7203; A61B 5/4839; A61B 5/16; A61B 5/7257

(57) ABSTRACT

A computer implemented method, system and software for detection of a biological condition including instructions for measuring a frequency modulated auditory evoked response of a subject and analyzing the frequency modulated auditory evoked response to detect the presence or absence of the biological condition in the subject. The biological condition can be an autism spectram disorder.

### TECHNICAL FIELD

The present invention generally relates to detecting and diagnosis of human disorders, for instance autism spectrum disorder and the like.

### BACKGROUND

Spectrum Disorders (ASD) or Autism refers to a complex range of developmental disorders that are characterized by impairments in language, difficulties with social interactions, and often rigid, repetitive and/or stereotypical behaviors and interests. The incidence of autism has increased over the last few decades, with as many as one in 88 children identified with ASD [1].

Retrospective studies indicate at least three distinct patterns of symptom onset in autism. In the most common form, symptoms of autism are evident early in infancy. In the second form, an initial period of typical development is followed by an unexpected cessation or significant slowing in the continued acquisition of communication and/or social skills and the child reaches a developmental plateau. In the third and perhaps most intriguing form, often referred to as regressive autism, a period of normal or near normal early development is followed by a cessation of all further development and loss of previously acquired communication and/or social skills, most often of both [2]. In this third form, developmental regression usually occurs between 15 to 30 months of age and can occur rapidly over a very few days, or more slowly in the course of several weeks or even months. Once development has regressed, such children typically follow the standard ASD developmental profile [3, 4]. Ozonoff et al. point out that although “more children may present with a regressive course than previously thought parent report methods do not capture this phenomenon well.”[5]. There may be confusion among early-starting slow regression, a developmental plateau, and frank, abrupt regression defined by rapid loss of previously achieved cognitive, language, communication, and behavioral milestones. Thus, anyone who studies patients with regressive autism must define carefully the parameters of regression applicable to the study.

ASD and in particular regressive autism have attracted worldwide attention. Prominent and abrupt loss of milestones typically results in referral to a child neurologist in order to search for potentially relevant etiological factors and, hopefully, for initiation of remediation. Neurological disorders associated with autism symptomatology include Fragile X syndrome, tuberous sclerosis, Rett's syndrome, mitochondrial disorders, and a multiplicity of genetic anomalies [6]. Regression associated with the Landau-Kleffner syndrome (LKS, an epileptic encephalopathy) may also present in a manner similar to R-ASD [7, 8]. An overnight sleep electroencephalogram (EEG) may be required to diagnose and/or rule out LKS, given that the finding of significant focal or generalized sleep-activated discharges is taken as diagnostic for LKS [9]. To date the cause of abrupt autistic regression often eludes identification, despite the fact that onset of regression may appear to follow a distinct event, such as trauma, illness, or immunization. This report focuses exclusively upon regressive autism of abrupt onset showing initial, obvious, rapid regression over a time period of a few days to maximally a few weeks, followed by continuing regression at a slower pace over the ensuing months until a stable low functioning state is reached. This phenomenon will be referred to as “R-ASD” in the current paper. It is probable that such abrupt regressions do not characterize the full third of autistic children, who are reported to “show regression” occurring across varying lengths of time [5].

The terminology used to describe regressive autism continues to be in flux. Given the similarity of presentation in children with R-ASD and children with early onset LKS the appellation “Landau-Kleffner Syndrome variant” (LKSv) was used and often retained even when a definitive LKS diagnosis had been ruled out. In such cases many epileptologists object to the “LKSv” terminology and increasingly utilize “regressive autism”, abbreviated as R-ASD, with focus upon the prominent autistic behavioral profile following regression. However, few R-ASD patients are evaluated early-on by “gold standard” measures such as the ADOS and ADI [10, 11]. Some behavioral neurologists object to the “autism” appellation despite the evidence that most such children eventually receive that diagnosis [3, 4]. The abbreviation R-ASD is used herein.

### SUMMARY OF THE INVENTION

Referring physicians often enquire about the utility of adrenal corticosteroids or glucocorticoids to treat patients with R-ASD. There are three possible justifications for this therapeutic approach. First, are the various similarities of R-ASD and LKS; many neurologists consider corticosteroids the ultimate treatment for LKS when anticonvulsants have failed [12, 13]. The R-ASD—LKS similarities considered include shared behavioral presentation [7, 8], strong overlap of genetic, genomic and molecular networks [14], and in some older R-ASD patients magnetoencephalography (MEG) detected seizure discharges localized to the bilateral superior temporal gyri (STG) [15], which are the same brain areas that show “active” EEG detected discharges in LKS [16]. Furthermore, in addition to LKS, several other forms of refractory epilepsy have been successfully treated using adrenal corticosteroids [17-19]. Given these similarities in underlying pathophysiology and genetics, and the potential of successful treatment, there is reason to hypothesize that the two syndromes may also share a positive response to corticosteroids. Second, it has been speculated that regressive autism might be an inflammatory or auto-immune disorder [20-22]. As corticosteroid administration constitutes an important treatment modality for the common auto-immune diseases [23], it is reasonable to consider steroids as potentially useful for R-ASD. A notable single case study demonstrated autistic language regression concomitant with an autoimmune disease; institution of steroid treatment of the autoimmune proliferative syndrome was associated with a parallel improvement in autistic symptoms, particularly in language [24]. Third, several additional case studies reported in the literature also indicate positive effects of steroids in children with R-ASD [15, 24, 25].

However, evaluating the effectiveness of a therapy (e.g. application of corticosteroids) to a behavioral or language disorder is difficult and hard to quantify. Accordingly, the inventors have identified a need to evaluate the effectiveness of administering corticosteroids as a therapy for the language related disorders disclosed herein. The inventors have discovered that utilization of a 4 Hz FMAER, for example, provides a reliable way of detecting improvements in autism spectrum disorders due to therapy. The 4 Hz FMAER produces a scalp recorded 4 Hz sine wave steady-state evoked response that arises in normal subjects from the bilateral STG as determined by source analysis. It is absent or distorted (non-sinusoidal appearance) in subjects with language disorders involving the STG. A normal FMAER, thus, would suggest a clinically apparent language abnormality that must have arisen from a cortical language processing disorder which originated outside of or beyond that of the STG.

Accordingly, systems and methods have been developed to test the response of a subject with an autism spectrum disorder or other related language disorders to application of corticosteroids or other therapies using EEG data. In some embodiments, this includes detecting changes in an FMAER after application of corticosteroids or other therapies to the subject to indicate an improvement in language or behavior aspects of the subject. The FMAER response those provides a quantitative mode to assess the effectiveness of therapies such as the application of corticosteroids in order to monitor the dosage and effectiveness in particular individuals.

This is particularly advantageous, because the use of the FMAER change may provide a quantitative way to assess correct dosages for each individual, and the responsiveness of each individual to different therapies. In addition, the EEG based method may be more quantitatively reliable than a verbal or other test administered by a health care professional which may be prone to error due to the requirements of subjective judgments of the professional. Additionally, this method may be quicker and easier to perform multiple times, without requiring the subject to repeat the same tests.

In one aspect, provided herein is a computer implemented method for detection of a biological condition, comprising: on a device having one or more processors and a memory storing one or more programs for execution by the one or more processors, the one or more programs including instructions for: measuring a frequency modulated auditory evoked response of a subject; and analyzing the frequency modulated auditory evoked response to detect the presence or absence of the biological condition in the subject.

In one embodiment of this aspect, the frequency modulated auditory evoked response is a 4 Hz frequency modulated auditory evoked response steady state response.

In another embodiment of this aspect, the biological condition is an autism spectrum disorder.

In another embodiment of this aspect, the autism spectrum disorder is a regressive form of the autism spectrum disorder.

In another embodiment of this aspect, the biological condition is Asperger's Syndrome.

In another embodiment of this aspect, the analyzing step comprises spectral noise analysis, and the frequency modulated auditory evoked response independently distinguishes between an absent response and a present but distorted and or noisy response. In another embodiment of this aspect, the analyzing step comprises a measurement of a 4 Hz magnitude and a 4 Hz signal to noise ratio taken during or after conclusion of an intervention.

In another embodiment of this aspect, the one or more programs further include instructions for: providing quantitative, scaled measures of improvement of superior temporal gyri and receptive language function.

In another embodiment of this aspect, the analyzing step comprises spectral analysis of a difference between standard and plus-minus signal averaging; and estimating stimulus generated noise content of an evoked response separate from a noise content from inadequate signal averaging.

In another aspect, provided herein is a computer system for detection of a biological condition, comprising: one or more processors; and memory to store: one or more programs, the one or more programs comprising instructions for: measuring a frequency modulated auditory evoked response of a subject; and analyzing the frequency modulated auditory evoked response to detect the presence or absence of the biological condition in the subject.

In one embodiment of this aspect, the frequency modulated auditory evoked response is a 4 Hz frequency modulated auditory evoked response steady state response.

In another embodiment of this aspect, the biological condition is an autism spectrum disorder.

In another embodiment of this aspect, the autism spectrum disorder is a regressive form of the autism spectrum disorder.

In another embodiment of this aspect, the biological condition is Asperger's Syndrome.

In another embodiment of this aspect, the analyzing step comprises spectral noise analysis, and the frequency modulated auditory evoked response independently distinguishes between an absent response and a present but distorted and or noisy response.

In another embodiment of this aspect, the analyzing step comprises a measurement of a 4 Hz magnitude and a 4 Hz signal to noise ratio taken during or after conclusion of an intervention.

In another embodiment of this aspect, the one or more programs further include instructions for: providing quantitative, scaled measures of improvement of superior temporal gyri and receptive language function.

In another embodiment of this aspect, the analyzing step comprises spectral analysis of a difference between standard and plus-minus signal averaging; and estimating stimulus generated noise content of an evoked response separate from a noise content from inadequate signal averaging.

In another aspect, provided herein is a non-transitory computer-readable storage medium storing one or more programs for detection of a biological condition, the one or more programs for execution by one or more processors of a computer system, the one or more programs comprising instructions for: measuring a frequency modulated auditory evoked response of a subject; and analyzing the frequency modulated auditory evoked response to detect the presence or absence of the biological condition in the subject.

In one embodiment of this aspect, the frequency modulated auditory evoked response is a 4 Hz frequency modulated auditory evoked response steady state response.

In another embodiment of this aspect, the biological condition is an autism spectrum disorder.

In another embodiment of this aspect, the autism spectrum disorder is a regressive form of the autism spectrum disorder.

In another embodiment of this aspect, the biological condition is Asperger's Syndrome.

In another embodiment of this aspect, the analyzing step comprises spectral noise analysis, and the frequency modulated auditory evoked response independently distinguishes between an absent response and a present but distorted and or noisy response.

In another embodiment of this aspect, the analyzing step comprises a measurement of a 4 Hz magnitude and a 4 Hz signal to noise ratio taken during or after conclusion of an intervention.

In another embodiment of this aspect, the one or more programs further include instructions for: providing quantitative, scaled measures of improvement of superior temporal gyri and receptive language function.

In another embodiment of this aspect, the analyzing step comprises spectral analysis of a difference between standard and plus-minus signal averaging; and estimating stimulus generated noise content of an evoked response separate from a noise content from inadequate signal averaging.

In another aspect, provided herein is a computer implemented method for detection of a biological condition, comprising: on a device having one or more processors and a memory storing one or more programs for execution by the one or more processors, the one or more programs including instructions for: measuring electroencephalography coherence of a subject; generating factor variables based on the measured electroencephalography coherence; and discriminating between and among neuro-psychiatric, neuro-developmental, and sensory processing disorders based on the factor variables.

In one embodiment of this aspect, the discriminating step comprises discriminating between and among normal controls, subjects with autism, and subjects with Asperger's Syndrome based on the factor variables.

In another embodiment of this aspect, the discriminating step comprises calculating a discriminant function based on the measured electroencephalography coherence.

In another embodiment of this aspect, the discriminating step comprises differentiation between low functioning autism and high functioning autism.

In another embodiment of this aspect, the one or more programs further comprise instructions for performing principal components analysis of the measured electroencephalography coherence.

In another embodiment of this aspect, a number of variables exceeds a number of subjects.

In another embodiment of this aspect, the discriminating step comprises calculating a discriminant function based on the measured electroencephalography coherence; and performing principal components analysis of the measured electroencephalography coherence.

In another aspect, provided herein is a computer system for detection of a biological condition, comprising: one or more processors; and memory to store: one or more programs, the one or more programs comprising instructions for: measuring electroencephalography coherence of a subject; generating factor variables based on the measured electroencephalography coherence; and discriminating between and among neuro-psychiatric, neuro-developmental, and sensory processing disorders based on the factor variables.

In one embodiment of this aspect, the discriminating step comprises discriminating between and among normal controls, subjects with autism, and subjects with Asperger's Syndrome based on the factor variables.

In another embodiment of this aspect, the discriminating step comprises calculating a discriminant function based on the measured electroencephalography coherence.

In another embodiment of this aspect, the discriminating step comprises differentiation between low functioning autism and high functioning autism.

In another embodiment of this aspect, the one or more programs further comprise instructions for performing principal components analysis of the measured electroencephalography coherence.

In another embodiment of this aspect, a number of variables exceeds a number of subjects.

In another embodiment of this aspect, the discriminating step comprises calculating a discriminant function based on the measured electroencephalography coherence; and performing principal components analysis of the measured electroencephalography coherence.

In another aspect, provided herein is a non-transitory computer-readable storage medium storing one or more programs for detection of a biological condition, the one or more programs for execution by one or more processors of a computer system, the one or more programs comprising instructions for: measuring electroencephalography coherence of a subject; generating factor variables based on the measured electroencephalography coherence; and discriminating between and among neuro-psychiatric, neuro-developmental, and sensory processing disorders based on the factor variables.

In one embodiment of this aspect, the discriminating step comprises discriminating between and among normal controls, subjects with autism, and subjects with Asperger's Syndrome based on the factor variables.

In another embodiment of this aspect, the discriminating step comprises calculating a discriminant function based on the measured electroencephalography coherence.

In another embodiment of this aspect, the discriminating step comprises differentiation between low functioning autism and high functioning autism.

In another embodiment of this aspect, the one or more programs further comprise instructions for performing principal components analysis of the measured electroencephalography coherence.

In another embodiment of this aspect, a number of variables exceeds a number of subjects.

In another embodiment of this aspect, the discriminating step comprises calculating a discriminant function based on the measured electroencephalography coherence; and performing principal components analysis of the measured electroencephalography coherence.

### BRIEF DESCRIPTION OF THE DRAWINGS

The accompanying drawings, which are incorporated into this specification, illustrate one or more exemplary embodiments of the inventions disclosed herein and, together with the detailed description, serve to explain the principles and exemplary implementations of these inventions. One of skill in the art will understand that the drawings are illustrative only, and that what is depicted therein may be adapted based on the text of the specification and the spirit and scope of the teachings herein.

In the drawings, where like reference numerals refer to like reference in the specification:

FIG. 1 depicts Frequency Modulated Auditory Evoked Response (FMAER) and Corresponding Fast Fourier Transform (FFT) in Regressive Autism, where FIG. 1A depicts data before steroid treatment and where FIG. 1B depicts data after steroid treatment; and

FIG. 2 depicts a computer device or system comprising one or more processors and a memory storing one or more programs for execution by the one or more processors.

FIG. 3 depicts a flow chart illustrating an example of a method for detecting an improvement of a condition.

### DETAILED DESCRIPTION

It should be understood that this invention is not limited to the particular methodology, protocols, etc., described herein and as such may vary. The terminology used herein is for the purpose of describing particular embodiments only, and is not intended to limit the scope of the present invention, which is defined solely by the claims.

As used herein and in the claims, the singular forms include the plural reference and vice versa unless the context clearly indicates otherwise. Other than in the operating examples, or where otherwise indicated, all numbers expressing quantities used herein should be understood as modified in all instances by the term “about.”

All publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Unless defined otherwise, all technical and scientific terms used herein have the same meaning as those commonly understood to one of ordinary skill in the art to which this invention pertains. Although any known methods, devices, and materials may be used in the practice or testing of the invention, the methods, devices, and materials in this regard are described herein.

### Some Selected Definitions

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments of the aspects described herein, and are not intended to limit the claimed invention, because the scope of the invention is limited only by the claims. Further, unless otherwise required by context, singular terms shall include pluralities and plural terms shall include the singular.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

Other than in the operating examples, or where otherwise indicated, all numbers expressing quantities used herein should be understood as modified in all instances by the term “about.” The term “about” when used in connection with percentages may mean±1%.

In embodiments of the disclosure, terms such as “about,” “approximately,” and “substantially” can include traditional rounding according to significant figures of the numerical value.

The singular terms “a,” “an,” and “the” include plural referents unless context clearly indicates otherwise. Similarly, the word “or” is intended to include “and” unless the context clearly indicates otherwise. Thus for example, references to “the method” includes one or more methods, and/or steps of the type described herein and/or which will become apparent to those persons skilled in the art upon reading this disclosure and so forth.

Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments of the aspects described herein, the subject is a mammal, e.g., a primate, e.g., a human. The terms, “patient” and “subject” are used interchangeably herein.

In some embodiments, the subject is a mammal. The mammal can be a human, non-human primate, mouse, rat, dog, cat, horse, or cow, but are not limited to these examples. Mammals other than humans can be advantageously used as subjects that represent animal models of disorders.

A subject can be one who has been previously diagnosed with or identified as suffering from or having a disease or disorder caused by any microbes or pathogens described herein. By way of example only, a subject can be diagnosed with sepsis, inflammatory diseases, or infections.

To the extent not already indicated, it will be understood by those of ordinary skill in the art that any one of the various embodiments herein described and illustrated may be further modified to incorporate features shown in any of the other embodiments disclosed herein.

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

### Neurophysiological Biomarkers

### The Frequency Modulated Auditory Evoked Response (FMAER) as a Biomarker

FMAER arises from the bilateral superior temporal gyri (STG) of normal subjects. The STG regions are regarded as the brain regions primarily engaged in the processing of receptive language.

In some embodiments, the FMAER is a steady state auditory evoked response presented as sound via earphones or loud-speakers to infants, children, and adults. In some embodiments, a 1000 to 2000 Hz constant auditory carrier can be frequency modulated (FM) at 10 Hz resulting in a warbling tone. The FM component can be slowly and gradually turned on and off via an enveloping 4 Hz sinusoid. A trial marker (TM), which can be locked to onset of the 4 Hz enveloping sine wave, can appear every second. Using the above trial markers, signal averaging {van Drongelen, 2011 #11309} can be performed on the electroencephalography (EEG) recorded from a normal subject's multichannel EEG recording during FMAER stimulation, and a 4 Hz scalp recorded sinusoidal so called “following” response can be detected utilizing a EEG recording system as disclosed herein. For instance, patients with absent, noisy or distorted FMAERs have been shown to have receptive language dysfunction.

The present inventor presently describes the use of the FMAER as a biomarker of receptive language disorder using quantitative techniques. For instance, spectral analyses of the steady state evoked response can be performed in order to evaluate the response. In other embodiments, the delay or phase difference between the evoked potential and the following response may be analyzed.

In language-normal subjects the final steady state evoked response results in a clear, isolated 4 Hz spectral component that provides a single numerical value (in μV²/Hz) for normal subjects. For a population of children with a history of regressive autism, the 4 Hz FMAER response appears absent across the scalp. When the children diagnosed with regressive autism are treated with a several months long course of oral corticosteroids, improvement in language and behavior are associated with a marked and statistically highly significant increase in the 4 Hz scalp recorded FMAER response magnitude. Furthermore, the FMAER 4 Hz response and the quantified language improvement response are highly correlated. Accordingly, in some embodiments, an increased or improved FMAER 4 Hz response is an indication of an improvement in language.

Additionally, the present inventor performed a noise analysis on the pre- and post-treatment FMAERs. The noise analysis consists of forming a signal-to-noise ratio of the 4 Hz wanted signal component to the unwanted side band components at adjacent 3, 5, 6, and 7 Hz, which are considered noise. The present inventor demonstrated that pre-treatment FMAER in the regressive autism sample shows a diminished 4 Hz response mixed within side band noise at 3, 5, and 6 Hz. However, at outcome after treatment, not only does the 4 Hz response magnitude greatly increase but the side-band noise components are significantly decreased as well. The noise analysis employed a plus-minus averaging technique {van Drongelen, 2011 #11309} to facilitate quantification and removal of any confounding noise component attributable to the signal averaging process itself.

Accordingly, the present invention is directed to at least the following: (1) a 4 Hz FMAER steady state response constitutes a unique, quantitative and non-invasive assessment of functional capability of an STG to process receptive language; (2) an FMAER independently distinguishes between an absent response and a present but distorted and or noisy response by spectral noise analysis; (3) measures of a 4 Hz magnitude and a 4 Hz signal to noise ratio (SNR), when taken during or after conclusion of an intervention provide quantitative, scaled measures of improvement of STG and receptive language function, i.e., non-invasive functional biomarkers; (4) two measurements of FMAER 4 Hz magnitude and 4 Hz SNR are of value and serve as functional biomarkers in a quantification of all types of receptive language deficiencies in children and adults; and (5) spectral analysis of a difference between standard and plus-minus signal averaging as a technique to estimate stimulus generated noise content of an evoked response separate from a noise content from inadequate signal averaging.

### EEG Spectral Coherence Factors Produced by Unrestricted Principal Components Analysis (PCA) and Defined by Discriminant Function Analysis (DSC) as a Biomarker

Spectral analysis of the ambient, background scalp EEG is a well-accepted technique to quantitate the differing frequencies that make up human EEG. Spectral coherence analysis is an additional technique that quantitates the similarity in spectral content, over time, between two selected EEG channels. Coherence is typically calculated separately, for each and every frequency contained within the EEG. Numerical coherence results in values that vary between 0 and 1. Coherence values are commonly taken as quantifying the functional, biological connectivity between two selected electrodes at a selected frequency. High coherence values indicate high connectivity and low coherence values signify low connectivity per electrode pair and frequency.

A problem that confronts statistical analyses of EEG coherence data arises from the numerous coherence values that are created per study. For example, for 24 EEG channels and 16 spectral frequencies, 4,416 unique values are produced. In typical studies it is common for the coherence data sets to be numerically reduced in number by selection based upon a priori assumptions. Thus, in an effort to accomplish data reduction and thereby limiting type 1 statistical errors (false positives), a priori pre-selection of specific coherences may inadvertently eliminate important coherence variables from subsequent analyses due to limited a priori knowledge and actually result in type 2 error (false negatives). The present inventor's published approach to this analytic quandary is to perform Principal Components Analysis (PCA) on the entire data set of 4,416 unique values to avoid the need for a priori variable reduction. PCA is an established technique which, on a single population, transforms input variables into a set of output variables called factors. The output factors represent a linear combination of the original variables, where input variables that are highly inter-correlated are mathematically compressed to form a single factor. Furthermore, PCA imposes order on the new factors based upon the amount of variance explained, i.e., the degree of information content contained in the factor given the original variables. Subsequent selection of the thus numerically reduced set of resulting EEG coherence factors is based upon the amount of information (variance) they collectively represent. For example, a 2012 publication {Duffy, 2012 #11624}, that showed successful separation by split-half replication of subjects with autism spectrum disorder from neuro-typical controls (normal children), utilized just 40 coherence factors, which accounted for over 50% of the total variance, i.e., 50% of the information, contained within the initial 4,416 coherence values. Separation of the population with autism from the normal population was accomplished by the use of Multivariate Discriminant Analysis (DSC) of the PCA-derived 40 coherence factors.

DSC is a statistical technique that acts upon all subjects once they have been individually represented by a set of variables, e.g., the coherence factors. DSC then separates two or more populations, thus represented, on the basis of a new variable. This new discriminant variable is created mathematically from the original variables and is optimized for the separation of the populations. DSC creates this new variable by a unique, linear combination of the original input variables. DSC also provides a set of rules by which the probability of each individual subject's assignment to one population or another is provided. This process was highly successful in the discrimination of the neurotypical subjects from the subjects with autism {Duffy, 2012 #11624}.

The PCA factors, created as described above, were used along with new PCA-DSC derived classification rules (also described above) and were successful in a study separating, i.e., contrasting, three groups, namely neurotypical children, children with autism, and children with Asperger's Syndrome {Duffy, 2013 #12230}.

In the above two population studies {Duffy, 2012 #11624; Duffy, 2013 #12230}, the present inventor demonstrated that the combination of PCA and DSC provides a useful methodology when based on EEG coherence factors. These studies successfully diagnosed, i.e., classified as distinct diagnoses, children with autism, children with Asperger's Syndrome and neurotypical children.

The present inventor presently discloses, for the first time, the specific software the present inventor developed and used to perform PCA, and the mathematical combination of the original EEG coherences used to form the coherence factors. Furthermore, the present inventor presently discloses, for the first time, the unique mathematics involved in the DSC process.

Accordingly, the present invention is further directed to at least the following: (6) a unique combination of EEG coherences to create factor variables used to discriminate between and among normal controls, subjects with autism, and subjects with Asperger's Syndrome; (7) unique mathematical details of a discriminant function used to differentiate above groups on a basis of EEG coherence factors, which can be used as a prospective biomarker; (8) a unique EEG coherence biomarker that distinguishes high from low functioning autism; and (9) unique software developed to perform PCA of large coherence data sets where there are more variables than subjects.

The creation of a biomarker is important since it may be used in a diagnostic process. However, it also provides a numeric and visual indication of an individual subject's position and/or movement along the biomarker axis. For example, subjects with autism move along the normal-autistic EEG coherence biomarker axis away from the autistic end towards the normal control end but may not pass the point where they may be re-classified as “normal”. A biomarker may provide an indication of the efficacy of a treatment designed to improve or cure a disease, short of indicating that the intervention has gone all the way to curing the disease. The biomarker may provide crucial information in the identification of the degree of efficacy of interventions, so that they could be discarded, tried again after improvements are made, and/or combined with other potentially successful interventions re-tried together.

Accordingly, the present invention is further directed to at least the following: (10) a PCA/DSC derived discriminant variable as a quantitative, scaled biomarker sensitive to a diagnosis of normal controls, children with autism, and children with Asperger's Syndrome; (11) PCA/DSC based EEG biomarkers as generally useful in distinguishing among childhood and adult neuro-psychiatric, neuro-developmental, and sensory processing disorders; and (12) a general use of DSC derived discriminant functions as biomarkers.

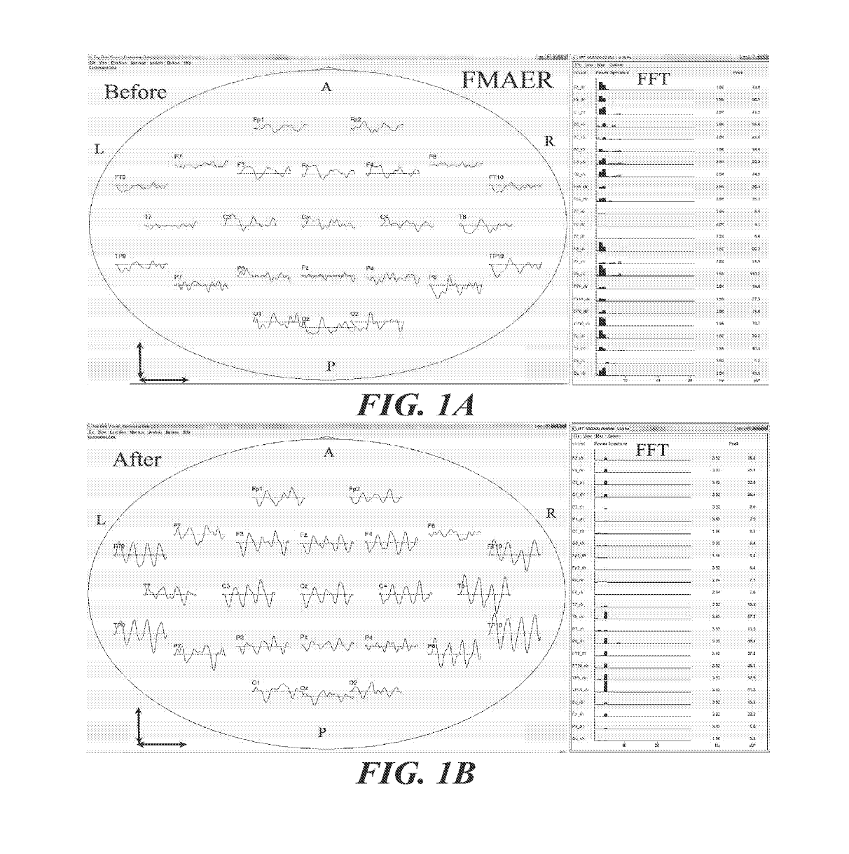

FIG. 1 illustrates a, 4 Hz FMAER waveform data before and after administration of a treatment, in this example steroid treatment. Illustrated in FIG. 1 are schematic ovals in vertex view with nose up, and left side of scalp to image left. The corresponding power spectra are shown to the immediate right. The top waveform and Fast Fourier Transform (FFT) displays were obtained prior to steroid administration. The bottom, corresponding displays were obtained after steroid administration. The vertical arrow to the lower left of each image represents 10 μV and the horizontal arrow beneath represents one second waveform length. The labels adjacent to the FMAER waveforms correspond to the standard EEG electrode 10-10 naming convention. Twenty-four electrodes' waveforms are shown. The FFT power spectral data horizontal axis covers the 0-30 Hz range. Note the near absent 4 Hz FMAER waveform response before and excellent 4 Hz waveform response after steroid administration. Note the spread of spectral power over many frequencies in the FFT display before (In FIG. 1A) which represents a distorted response. This contrasts to the nearly perfect 4 Hz response after steroid treatment (In FIG. 1B) which shows little spectral spread (little distortion).

In FIG. 1, for the vertex view display, waveforms are shown overlying their standard “10-10” locations. For the FFT graphs, channel order from top to bottom is: F3, F4, C3, C4, P3, P4, O1, O2, Fp1, Fp2, F7, F8, T7, T8, P7, P8, FT9, FT10, TP9, TP10, Fz, Cz, Pz, Oz. The common average reference is utilized for the displayed data (a reference free or “rfr” technique) [10].

In FIG. 1, abbreviations are used as follows: A=anterior, P=posterior, L=left, R=right, FMAER=4 Hz frequency modulated auditory evoked response, FFT=fast Fourier transform—power spectrum analysis shown as μV²/Hz, μV=microvolt, and Hz=Hertz or cycles per second.

FIG. 1 illustrates an example of one STAR group subject's relevant FMAER and associated spectral changes from before to after steroid administration: Note the absence of a clear 4 Hz sine wave following response before treatment in contrast to the excellent 4 Hz sine wave following response after treatment. The “before treatment” fast Fourier transform (FFT) power spectrum response furthermore illustrates that the input 4 Hz stimulus appears at the cortex and demonstrates power at many different frequencies aside from the expected 4 Hz input response. This occurrence of spurious response frequencies in output to a single frequency input represents response distortion [37]. It is visualized in the FMAER waveforms by their non-sinusoidal appearance and in the FFT by the spread across the spectrum away from the primary 4 Hz driving frequency. Note, however, that after steroids the FMAER waveform was sinusoidal at 4 Hz and the FFT showed a well-aligned response at the expected 4 Hz frequency without spectral spread, i.e., without distortion. All STAR subjects uniformly demonstrated such reduction of distortion after steroid treatment, while this change was not observed in the NSA group

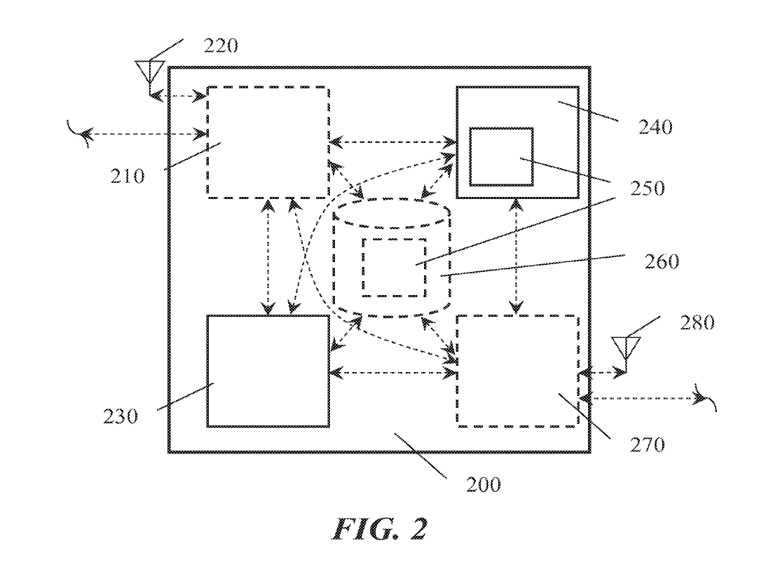

FIG. 2 depicts a computer device or system 200 comprising one or more processors 230 and a memory 240 storing one or more programs 250 for execution by the one or more processors 230. In some embodiments, the device or computer system 200 can further comprise a non-transitory computer-readable storage medium 260 storing the one or more programs 250 for execution by the one or more processors 230 of the device or computer system 200.

In some embodiments, the device or computer system 200 can further comprise one or more input devices 210, which can be configured to send or receive information to or from any one from the group consisting of: an external device (not shown), the one or more processors 230, the memory 240, the non-transitory computer-readable storage medium 260, and one or more output devices 270. The one or more input devices 210 can be configured to wirelessly send or receive information to or from the external device via a means for wireless communication, such as an antenna 220, a transceiver (not shown) or the like.

In some embodiments, the device or computer system 200 can further comprise one or more output devices 270, which can be configured to send or receive information to or from any one from the group consisting of: an external device (not shown), the one or more input devices 210, the one or more processors 230, the memory 240, and the non-transitory computer-readable storage medium 260. The one or more output devices 270 can be configured to wirelessly send or receive information to or from the external device via a means for wireless communication, such as an antenna 280, a transceiver (not shown) or the like.

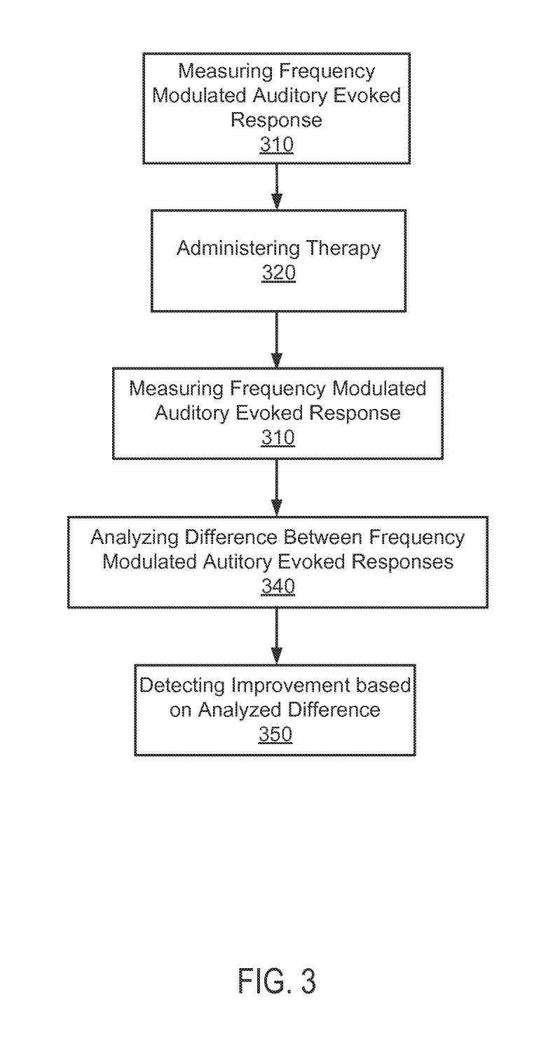

FIG. 3 depicts a method of detecting an improvement in behavior or language of a subject after administering a treatment to the subject. The method includes the step of measuring a frequency modulated auditory evoked response 310 of a subject. In some embodiments, that subject may also have an autism spectrum disorder, a regressive autism spectrum disorder or other disorder that includes a distorted or diminished response to the frequency modulated auditory stimulus as disclosed herein.

In some embodiments, a 4 Hz frequency modulated evoked response (FMAER), may be utilized which arises from language-relevant cortex of the superior temporal gyrus (STG), and upon EEG background activity, language, and behavior. Accordingly, audio speakers may be placed in proximity to the patient for broadcasting the audio stimulus to the patient and may be incorporated in systems and methods that monitor and apply the FMAER.

For instance, the FMAER may be formed from a carrier sine wave at 1000 Hz frequency modulated by a slower 10 Hz sine wave causing the frequency of the carrier wave to shift (“deviate”) between 960 and 1060 Hz at the 10 Hz rate, producing a warbling tone. In other embodiments, a 1000 Hz frequency may be modulated by a 9 Hz, or 11 Hz, or other frequency. In some embodiments, the 10 Hz sine wave may then be amplitude modulated by a slower 4 Hz sine wave such that the warbling (FM modulation) can be sinusoidally turned on and off (AM modulated) at a 4 Hz rate. In other embodiments, the 10 Hz sine wave amy be amplitude modulated by a 3 Hz, 5 Hz, or 6 Hz sine wave. This process caused the 10 Hz “warbling” of the 1000 Hz sine wave carrier to be sinusoidally turned fully on and off (100% modulation) at 4 Hz or other frequencies.

In some embodiments, by setting a trigger pulse to the start of each second of 4 Hz signal, signal averaging may be performed in order to obtain a 4 Hz steady-state FMAER time locked to the 4 Hz AM modulation of the 10 Hz FM modulation, i.e., to the turning on and off of the FM. Alternatively, this may be performed with respect to other frequencies.

In some embodiments, between 500-1000 trigger pulses may be averaged over an epoch of 1000 msec using BESA software (BESA GmbH, Freihamer Str. 18, 82116 Gräfelfing, Germany). After applications of the FMAER, the one-second-long steady state FMAER tracing from neurotypical subjects manifests as a 4 Hz sine wave. In some embodiments, the stimulus' sound pressure level may applied approximately 78 db Sound Pressure Level (SPL), measured at the ears and was delivered either by earphones or by nearby bilateral speakers depending upon the environment and subject preference and tolerance. In other embodiments, the sound pressure level may be other reasonable pressures from 60-80 db, or other levels.

The second step in FIG. 3 includes administering a therapy 320 to the subject. In some embodiments, administering a therapy 320 may include administering a dose of corticosteroid to a subject. In other embodiments, an anti-inflammatory substance may be administered, cognitive therapy may be performed, or other therapy may be applied. Then, the FMAER is measured 310 following therapy, utilizing the same FMAER or excitation stimulus as in the first step to maintain consistency. After both responses are captured, the differences between the two FMAER before and after administration of the therapy may be analyzed 340 to detect an improvement based on the analyzed difference 350.

In order to analyze some responses, a FFT or other transformation may be performed to determine the frequencies present in the responses, and the associated amplitudes of those frequencies. In some embodiments, frequencies present outside the stimulation frequency (e.g., 4 Hz) may indicate abnormal language processing. In some embodiments, after applying a FFT to both signals, the average power for each frequency range above a threshold may be subtracted to output a before and after difference. In some embodiments, an increase in power of the 4 Hz or applied frequency and/or a decrease in power of adjacent frequencies (that indicate a noisy response indicative of a language processing disorder) may be detected to indicate an improvement in language or behavior related to the autism condition. Accordingly, the system may record the average power differences and provide a score that shows the improvement, and may indicate whether additional treatment may be necessary and the quantity of additional treatment. For instance, if the dosage of steroids illustrated a 30% improvement over the maximum or full improvement, a corresponding increase in dosage, or continuing of the same dose over time may be applied.

In some embodiments, a plus-minus average and Vrms technique may be utilized. By creating the root-mean-square voltage (Vrms) of a plus-minus average one can compare this Vrms to the Vrms of the standard average. If there is no evoked response the standard and plus-minus averages generally show nearly the same Vrms values. If there is an evoked response the standard average typically produces a Vrms greater than does the plus-minus average. Since the plus-minus and the standard averages have the same noise distribution, spectral analysis of the plus-minus average can be subtracted from that of the standard average in order to estimate the spectral distribution of signal added in response to the stimulus. An advantage to the plus-minus technique is that noise is estimated on the very same EEG segments that are averaged in order to produce the FMAER.

### Example 1

The following example is provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

In this example, subjects with regressive autism were utilized to determine whether application of a corticosteroid would improve language function and show an improved 4 Hz FMAER. This study examines a clinical sample of children with R-ASD and treated with corticosteroids (average 9.1+/−3.3 months) in order to assess the effects of corticosteroid therapy in R-ASD upon a steady state evoked response, namely the 4 Hz frequency modulated evoked response (FMAER), which arises from language-relevant cortex of the superior temporal gyrus (STG), and upon EEG background activity, language, and behavior. A pharmacologically untreated clinical convenience sample of children with ASD served as controls.

Subjects

Steroid Treated Autism with Regression (STAR): The target group of study subjects was limited to children aged 3-5 years old, diagnosed as having autism with a historically documented period of regression at onset. Regression was defined as the loss of age appropriate language, communication, cognitive ability, and behavior determined by the referring physician and confirmed by the treating neurologist. Patients with a slowly developing plateau or a very slow regression over a prolonged period of time, as opposed to an abrupt regression, were not included in the STAR group. Furthermore, the target group was restricted to those patients, who were clinically treated with corticosteroids (STAR group) subsequent to an initial neurophysiological study that showed an absent or distorted FMAER, and who all had a second neurophysiological study after the treatment period was concluded, i.e., after at least six and not more than 36 months.

Non-Steroid-Treated Autism (NSA): The comparison group of subjects was similarly selected from 3-5 year old children with a diagnosis of autism with or without a documented regression. They had not received steroid treatment at any time during the study period (NSA), nor had they received any other pharmacological treatment. Similarly to the STAR group children, they had two sequential neurophysiological studies, which were separated by at least six and not more than 36 months, each of which contained an EEG and an FMAER [16]; the first FMAER study showed an absent or distorted FMAER wave form. Clinical treatment of the NSA group children, as for the STAR group, was in every case a decision between the respective treating physician and the parent(s), responsible for the child's care.

Subject Exclusion Criteria: For both, the STAR and NSA study groups, exclusion criteria included: (1) Co-morbid neurologic syndromes that may present with autistic features (for example, Rett's, Angelman's and Fragile X syndromes, tuberous sclerosis, or mitochondrial disorders); (2) clinical seizure disorders or EEG reports suggestive of an active seizure disorder or epileptic encephalopathy such as the Landau-Kleffner syndrome or prominent discharge activation during drowsiness or sleep (Note: occasional EEG spikes were not an exclusion criterion) [26]; (3) a primary diagnosis of global developmental delay (GDD) or developmental dysphasia; (4) other concurrent neurological disease processes that might induce EEG alteration (for example, hydrocephalus, hemiparesis or known syndromes affecting brain development); (5) significant primary sensory disorders, for example, blindness and/or deafness; (6) use of anticonvulsants (e.g., valproate, levetiracetam) at the time of the first study (Note: Prior failed use of a medication such as valproate did not constitute an exclusion criterion. [27]); (7) inadequate or incomplete clinical information; and (8) a normal, initial FMAER test result. All subjects in the DNL ASD data base, who fulfilled the study's in-and exclusionary criteria were included in the study sample. This yielded a study population of 20 target (STAR) and 24 comparison group (NSA) subjects.

Steroid Treatment STAR Patients: Oral prednisolone (Prelone™ or Orapred™) was administered by the parents on a daily basis at 2 mg/kg/day. Dosage was occasionally down-adjusted on the basis of minor complications.

Data Acquisition

All subjects' electrophysiological data utilized in this study were gathered from 30 scalp channels via gold cup electrodes applied with collodion after careful measurement. A 31st channel carried the FMAER trial marker. A 32nd channel carried eye movement and blink artifact information. Data were digitized at 256 Hz after amplification by a Cardionics™ 32 channel EEG amplifier (Cardionics Inc. 910 Baystar Blvd, Webster, Tex. 77598 USA) set to 1-100 Hz pass band. The 30 EEG scalp channels used included the following: FP1, FP2, F7, F3, FZ, F4, F8, FC5, FC1, FC2, FC6, T7, C3, CZ, C4, T8, CP5, CP1, CP2, CP6, P7, P3, PZ, P4, P8, O1, OZ, O2, TP9, and TP10 [28]. Data for the FMAER were gathered over 5-20 minutes with additional time allowed for rest breaks as indicated. The patient and a parent, when behaviorally indicated, were together within view of the technologist through a one-way mirror window in a sound-shielded room, adjacent to the recording equipment. Off-line, the EEG data and accompanying trial markers were visually evaluated and epochs containing excessive eye-blink, muscle and movement artifact were marked for removal from subsequent analysis. All 30 channel data were visually inspected for EEG abnormalities and for creating the common average reference. For the current study, FMAER analysis was restricted to 14 active scalp electrodes: F7, F8, T7, T8, P7, P8, TP9, TP10, F3, F4, C3, C4, P3, and P4.

Frequency Modulated Auditory Evoked Response (FMAER): Language comprehension requires decoding of rapidly changing speech streams; detection of FM within speech has been hypothesized as essential for accurate phoneme detection and word comprehension [16]. The FMAER was developed as a steady-state EP to assess the brain's response to rapid changes in the frequency modulation (FM) of an applied auditory stimulus in extension of and based upon the pioneering work of Green, Stefanatos and others [15, 29-33]. By source analysis, the FMAER has been shown to arise bilaterally from the superior temporal gyri (STG) of neuro-typical subjects. The orientation of the STG source dipoles in normal subjects is such that they each point at one end of the dipole toward the midline frontal region and at the other end to the ipsilateral inferior, posterior temporal region. The best electrodes to record an ipsilateral signal when using the common average reference are TP9 (left posterior-inferior temporal for left source dipole) and TP10 (right posterior-inferior temporal for right source dipole). Midline frontal electrodes (FZ, FC1, FC2) usually manifest bilateral overlapping source dipole projection. The central electrodes (C3-left central, C4-right central) give the best view of the corresponding hemisphere's source dipole's superior-anterior projection. The FMAER may be normal, or as in pathology, of low amplitude, distorted, or absent. In rare cases of pathology the FMAER source location may be outside the usual STG location and/or the source dipole may point in unexpected directions. [16].

As previously detailed [16], the FMAER was formed from a carrier sine wave at 1000 Hz frequency modulated by a slower 10 Hz sine wave causing the frequency of the carrier wave to shift (“deviate”) between 960 and 1060 Hz at the 10 Hz rate, producing a warbling tone. The 10 Hz sine wave was then amplitude modulated by a slower 4 Hz sine wave such that the warbling (FM modulation) was sinusoidally turned on and off (AM modulated) at the 4 Hz rate. This process caused the 10 Hz “warbling” of the 1000 Hz sine wave carrier to be sinusoidally turned fully on and off (100% modulation) at 4 Hz. By setting a trigger pulse to the start of each second of 4 Hz signal, signal averaging was performed in order to obtain the 4 Hz steady-state FMAER—time locked to the 4 Hz AM modulation of the 10 Hz FM modulation, i.e., to the turning on and off of the FM. Between 500-1000 trigger pulses were averaged over an epoch of 1000 msec using BESA software (BESA GmbH, Freihamer Str. 18, 82116 Gräfelfing, Germany). The one-second-long steady state FMAER tracing from neurotypical subjects manifests a 4 Hz sine wave. The stimulus' sound pressure level was held at approximately 78db Sound Pressure Level (SPL), measured at the ears and was delivered either by earphones or by nearby bilateral speakers depending upon the environment and subject preference and tolerance. At time of clinical study, FMAERs were initially formed from successive thirds of all stimuli which, when separately evaluated, allowed assessment of response consistency. If responses were similar across all three thirds, a global average was formed for interpretation. If such consistency was not observed, more data were collected to improve the signal to noise ratio. The Chirp2™ Signal Generator (Mind Spark Inc., 172 Washington St, Newton, Mass. 02458 USA), a small stand-alone battery operated device, was employed to perform all aspects of the FMAER from signal generation through trial marker formation.

FMAER data were viewed on BESA software using the common average reference since mastoid/ears references induce artifactual spatial localization. Each subject's response was visually reviewed for both hemispheres. The normal response consisted of a clear 4 Hz sine wave. An abnormal response varied from absence of any obvious response to a distinctly non-sinusoidal response suggesting a mix of multiple frequencies. In order to quantify responses for analytic purposes, power spectral analysis was performed on the FMAER traces (BESA software) and the resulting 4 Hz spectral value was utilized as the primary quantitative measure of the brain's response [16].

Noise Analysis: The steady state 4 Hz FMAER, when “absent” by visual inspection rarely presents as a simple flat-line display. Typically a low amplitude epoch of apparently random noise is visualized that does not appear to contain an obvious 4 Hz component. Such a noisy response reflects four possibilities: (1) There is no 4 Hz response and the noise reflects incomplete signal averaging; (2) There is a low amplitude 4 Hz response, which is masked by noise from incomplete signal averaging; (3) The response is distorted, showing frequencies adjacent to 4 Hz (side-band distortion) which causes the response to appear non-sinusoidal or noisy; or (4) A combination of the above three possibilities might be identified. In order to assess these possibilities, in keeping with established techniques, a plus-minus averaging technique was utilized “in which measurements from every other trial are inverted prior to creating the averaged result which removes the consistent signal component by the alternating addition and subtraction (and) the noise component is the same as that produced by the standard average [34](page 61). By creating the root-mean-square voltage (Vrms) of a plus-minus average one can compare this Vrms to the Vrms of the standard average. If there is no evoked response the standard and plus-minus averages typically show nearly the same Vrms values. If there is an evoked response of any sort the standard average typically produces a Vrms greater than does the plus-minus average. Since the plus-minus and the standard averages have the same noise distribution, spectral analysis of the plus-minus average can be subtracted from that of the standard average in order to estimate the spectral distribution of signal added in response to the stimulus. An advantage to the plus-minus technique is that noise is estimated on the very same EEG segments that are averaged in order to produce the FMAER. Software was developed in-house to perform plus-minus averaging and to create Vrms data.

For the current study noise analysis was limited to the STAR population's FMAER data from the two left hemisphere electrodes (TP9, C3), which manifested the most significant pre- and post-treatment FMAER difference at 4 Hz (Table 4B). The locations roughly correspond to the maximum scalp projection of the superior and inferior aspects of the typical FMAER dipole generator located in the left superior temporal gyrus [16]. At the time of signal averaging, the EEG was additionally band pass filtered from 1-12 Hz. An estimate of the FMAER response was taken at 4 Hz and an estimate of “side-band noise” was taken as the average of the spectral data at 2, 3, 5, 6, and 7 Hz at both study points. To start, the Vrms of the standard average and plus-minus average for the first time point (before steroids) data were compared to determine if any added component was evident in the standard average. Next, and separately for the first and second study time point, spectral analysis was performed independently on the standard FMAER average as well as on the plus-minus average. The plus-minus average spectral results were then subtracted from the standard spectral results at each study point separately. The resulting spectral difference result estimated the portion of spectral signal attributable to the stimulus, after removal of the best estimate of spectral background noise.

Subjective Evaluation of EEG Abnormality: All first and second EEG studies were visually reassessed by a pediatric electroencephalographer blinded to subject history and group identity. All EEGs were reviewed in randomly selected order with respect to group and study order. Each EEG was scored as showing in ascending order of abnormality (if present): paroxysmal sharp theta, sharp waves, and spike or spike wave discharges. After this “blinded” estimation was completed, EEG results were sorted into first and second EEG study order, without group identity; subsequently the electroencephalographer compared each subject's two sequential EEGs and classified their scores as showing “worsening”, “no change”, or “improvement” between the first and second study. For example, a subject showing “spikes” on the first study and “no spikes” on the second study would be considered as “improved”.

Language Assessment, STAR Group: STAR group subjects were followed by their child neurologists using a clinical language assessment, referred to as the “Clinical Language Status Questionnaire (CLSQ)” which was developed in-house well prior to the current study. It is as yet unpublished. The assessment was designed to assist pediatric neurologists involved in the pharmacological treatment of neurological disorders, in estimating language progress or lack thereof—from a child's parent(s)' report and the clinician's direct assessment at the time of a clinical visit. It involves evaluation of both the child's current best expressive and receptive language performance. In the current study, documentation of lack of response improvement or plateau, along with assessment of medical complication(s), constituted the primary evidence used for treatment discontinuation. Table 1 shows the CLSQ score definitions for expressive and receptive language performance. The term “Appears normal” (Table 1) was defined as the clinician's and family's report of normal, age-appropriate speech. None of the children in this study received this score. “Appears nearly normal” was defined as context and age appropriate speech with evidence of mispronunciations, poor or odd word choice, unusual fluency or unusually sparseness, unusual grammatical errors such as errors in tense, pronoun gender match and/or pluralization, and/or requirement of adult speech simplification in order to assure comprehension relative to age expectancy. The difference between “short meaningful 1-2 word phrases” and “meaningful 3+ word phrases” is self-explanatory. Although many well developed language tests exist, as documented by the American Speech-Language-Hearing Association (www.asha.org/assessments.aspx), most tend to be detailed yet too coarsely grained to capture this population's limited language range. They are also typically too time-consuming for repeated administration by neurologists during recurring, clinical check-in visits. In contrast, the CLSQ may be completed in less than ten minutes. An additional advantage of the CLSQ for the current study was its comprehensiveness in capturing the full—while limited—range of language change observed in this patient population. Parents are shown short phrases (without the numerical scores) and asked to identify their child's current relevant expressive and receptive status. The neurologist stands by to clarify, assist, and help resolve two caregivers' divergent opinions. Neurologists using the CLSQ are sensitive to the language performance definitions in question. The study subjects' CLSQ score assignments occurred without prior knowledge of the current study.

TABLE 1
Clinical Language Status Questionnaire (CLSQ)

| Expressive Score | Receptive Score |
|---|---|
| 10 Appears normal | 10 Seems normal |
| 9 Normal but dysarthric | 9 Nearly normal receptively |
| 9 Nearly normal expressively | 9 Responds to incidental language |
| 9 1-3 word sentence | 9 Responds to multiple (>2) part requests |
| 9 Produces meaningful (>2 word) phrases | 8 Responds to two part requests |
| 8 Produces meaningful 1-2 word phrases | 6 Responds to one part requests |
| 7 Produces single words on own initiative | 4 Responds to words without gestures |
| 5 Mimics words strings without meaning | 2 Responds to words with gestures |
| 4 Produces meaningless words | 1 Responds better to voices than to noises |
| 3 Only sings words | 0 Responds better to noises than to voices |
| 1 Produces word-like meaningless sounds | 0 Acts deaf |
| 1 Babbles, no words | |
| 0 Makes noises, or only screams | |
| 0 Mute | |

Receptive Language Scoring
Ask the parent(s): “In the course of the last month what is the most complex
spoken language, given without helpful gestures, that you know your child understands and
may respond to but need not respond to every time.”
Ask for discrete examples and match to 11 shown possibilities. We are after the
highest level response of which the parents are certain. Parents may be shown the
alternative choices but not the associated scores.
Expressive Language Scoring
Ask the parent(s): “In the course of the last month what is the most complex
spoken language you have heard your child produce.” Again we are after the highest
level of language production of which the parents are certain. Match to one of the 14
shown possibilities. Parents may be given the alternative choices but not the associated
scores.

Language Assessment, NSA Group: Since the CLSQ was not employed for those pediatric populations who did not receive pharmacological intervention(s), the NSA group subjects lacked the CLSQ scores. Their language status was estimated retrospectively on the basis of the clinical assessment of expressive and receptive language abilities performed within the standard comprehensive neurological evaluation included in every office visit. The clinical reports closest in time to the two neurophysiological studies were utilized to score separately receptive and expressive language performance and to assign a change score from the initial to the second visit. The change score was scaled from minus (−) 2 to plus (+) 2 as follows: −2=marked worsening; −1=some worsening; 0=no change; +1=some improvement; +2=marked improvement. In the case a clinician failed to distinguish between receptive and expressive language performance the same score was assigned to both categories. Failure to comment on language performance at all resulted in exclusion of the subject from consideration for the current study. No subjects were excluded on this basis. NSA subjects' language scoring was performed well before the current study was undertaken and without knowledge as to which particular subject might be included in the study. Scorers had no knowledge of the current study's goals or design, the future group status of subjects, or the subjects' FMAER results. Approximately three times as many children's language reports were scored retrospectively as were declared eligible for inclusion in the study.

Behavioral Assessment: The DSM-IV criteria for Autistic Disorder (299.0) [35], aside from employment for diagnosis, were scaled in terms of severity for each STAR group child and the sum of the scaled scores was assigned as behavioral score. The direction of scaling was defined such that the higher the score was, the more severe the behavioral manifestations of ASD were. The symptom items and scaling are described in Table 2. Note, that item A2b) was omitted from the total score since none of the study children demonstrated “adequate speech”. Summed scaled item scores were assigned in order to quantify behavioral change in analogy to language change. For the current study scores were selected for the office visit nearest to the first and the second EEG/FMAER studies respectively. Scorers had no knowledge of the current study's goals or design, the future group status of subjects, or the subjects' FMAER results. The NSA group subjects lacked the DSM-IV based behavior scores.

TABLE 2
DSM-IV Criteria for Autism Disorder, and Scoring

Criteria:
A subject must have a total of six (or more) items from A1, A2, and A3. with at least two
from A1 and at least one each from A2 and A3

| | |
|---|---|
| A1. | Qualitative impairment in social interaction, as manifested by at least two of the |
| | following: |

| | |
|---|---|
| A1a) | Marked impairment in the use of multiple nonverbal behaviors such as eye- |
| | to-eye gaze, facial expression, body postures, and gestures to regulate social |
| | interaction. |
| A1b) | Failure to develop peer relationships appropriate to developmental level |
| A1c) | Lack of spontaneous seeking to share enjoyment, interests, or achievements |
| | with other people (e.g., by lack of showing, bringing, or pointing out objects |
| | of interest) |
| A1d) | Lack of social or emotional reciprocity |

| | |
|---|---|
| A2. | Qualitative impairments in communication as manifested by at least one of the |
| | following: |

| | |
|---|---|
| A2a) | Delay in, or total lack of, the development of spoken language (not |
| | accompanied by an attempt to compensate through alternative modes of |
| | communication such as gesture or mine) |
| A2b) | In individuals with adequate speech, marked impairment in the ability to |
| | initiate or sustain a conversation with others (omitted from scoring - see |
| | text) |
| A2c) | Stereotyped and repetitive use of language or idiosyncratic language |
| A2d) | Lack of varied, spontaneous make-believe play or social imitative play |
| | appropriate to developmental level |

| | |
|---|---|
| A3. | Restricted repetitive and stereotyped patterns of behavior, interests and activities, as |
| | manifested by at least one of the following: |

| | |
|---|---|
| A3a) | Encompassing preoccupation with one or more stereotyped patterns of |
| | behavior and restricted patterns of interest that is abnormal either in |
| | intensity or focus |
| A3b) | Apparently inflexible adherence to specific, nonfunctional routines or |
| | interests |
| A3c) | Stereotyped and repetitive motor mannerisms (e.g., hand or finger flapping |
| | or twisting, or complex whole-body movements) |
| A3d) | Persistent preoccupation with parts of objects |

| | |
|---|---|
| B. | A subject must show delays or abnormal functioning in at least one of the following |
| | areas, with onset prior to 3 years: |

| | |
|---|---|
| B1) | Social interaction |
| B2) | Language as used in social communication |
| B3) | Symbolic or imaginative play |

Graded Scoring - Each Item: (Item A2b omitted)

0 = absent
1 = possibly or very mildly present
2 = definitely present
3 = a very dominant characteristic
Overall score = average of 14 scored items

Data Analysis

The BMDP2007™ statistical package (Statistical Solutions, Stonehill Corporate Center, Suite 104, 999 Broadway, Saugus, M A 01906 USA) [36] was utilized for standard statistical analyses. Program 2D (P2D) was used for data description, Program 3D (P3D) for paired t-tests, and Program 2R (P2R) for multiple regression. Fisher's exact test for 2×2 tables utilized an online Graph Pad™ program. Fisher's exact test for 2×3 tables with Freeman-Halton extension utilized the online Statistical Calculators™ program.

Results

Group Demographics: The STAR group's sample size was 20 and the NSA group's 24 subjects (see Table 3 for demographics). The means and standard deviations (SD) of the ages for the two groups at the time of the first neurophysiologic study were 3.91 (1.25) and 4.52 (1.80) years respectively and the mean and SD time between the first and second studies was 2.14 (1.61) and 1.90 (0.99) years respectively. Neither age at first study nor interval between studies differed statistically between the two groups. All STAR group subjects manifested language and behavior regression sufficiently abrupt to allow parents to date onset to within a few days to maximally a few weeks. Mean and SD STAR group age at regression onset was 18.93 (9.93) months and mean length of regression to a point of relative stability was 20.05 (12.7) weeks. Mean and SD of steroid treatment length was 9.125 (3.26) months, with a range from 4 to 14 months. The two groups did not statistically differ by Fisher Exact test regarding gender or handedness (Table 3). By definition the STAR group was entirely (20 of 20) comprised of children with regressive autism. The NSA group contained, by chance, several children with regressive autism (7 of 24) (Table 3). This difference was significant by Fisher's Exact test, p=0.0001.

TABLE 3
Group demographics

| | STAR Group (n = 20) | NSA Group (n = 24) | t-test | p |
|---|---|---|---|---|
| Age at first study | 3.909 +/− 1.248 | 4.522 +/− 1.800 | 1.29 | n.s. |
| (years) | | | | |
| Time between | 2.136 +/− 1.609 | 1.904 +/− 0.989 | 0.49 | n.s. |
| studies (years) | | | | |
| Age at regression | 18.925 +/− 9.928 | | | |
| (months) | | | | |
| Length of regression | 20.053 +/− 12.70 | | | |
| (weeks) | | | | |
| Length of treatment | 9.125 +/− 3.26 | | | |
| (months) | | | | |
| Gender | 18 males, 2 females | 18 males, 6 females | | n.s. |
| Handedness | 18 right, 2 left | 23 right, 1 left | | n.s. |
| Subjects with history | 20 of 20 | 7 of 24 | | 0.0001 |
| of regression (n) | | | | |

Change in FMAER Between time 1 and time 2, Subsequent to Steroid Treatment: As shown in Table 4A, for the STAR group nine of 14 electrode measurements of the FMAER's 4 Hz response manifested a significant change, measured by matched-paired t-test, between study values before and after steroid therapy. The biggest change was identified at the left posterior-inferior temporal electrode TP9 (p=0.0037). In contrast, not one of the 14 analogous FMAER spectral measures compared between time 1 and time 2 for the NSA group was statistically significant. All NSA group members demonstrated an increase in 4 Hz magnitude from study 1 to study 2; this did not exceed the expected change with age. None of the NSA group's study 1 to study 2 differences was significant at any of the electrode sites measured.

TABLE 4A
Time 1 to Time 2 difference of the 4 Hz FMAER, paired t-tests

| | STAR Group | | | NSA Group | | |
|---|---|---|---|---|---|---|
| | Mean | | | Mean | | |
| Electrode | Diff. | T | p | Diff. | T | p |
| F3 | 47.18 | 2.57 | 0.0188 | 16.83 | 1.17 | n.s. |
| C3 | 24.15 | 3.02 | 0.0070 | 15.45 | 1.35 | n.s. |
| P3 | 10.49 | 1.25 | n.s. | 3.23 | 1.04 | n.s. |
| F7 | 3.38 | 0.51 | n.s. | 3.26 | 0.72 | n.s. |
| T7 | 15.49 | 2.83 | 0.0106 | 6.31 | 0.83 | n.s. |
| P7 | 65.08 | 2.37 | 0.0283 | 24.69 | 1.71 | n.s. |
| TP9 | 56.91 | 3.31 | 0.0037 | 31.77 | 1.78 | n.s. |
| F4 | 64.31 | 2.41 | 0.0260 | 20.89 | 1.21 | n.s. |
| C4 | 22.66 | 3.12 | 0.0056 | 13.48 | 1.15 | n.s. |
| P4 | 10.52 | 1.96 | n.s. | 3.12 | 0.59 | n.s. |
| F8 | 18.47 | 1.44 | n.s. | 8.02 | 0.95 | n.s. |
| T8 | 30.60 | 1.87 | n.s. | 1.08 | 0.07 | n.s. |
| P8 | 77.83 | 2.86 | 0.0100 | 35.87 | 1.36 | n.s. |
| TP10 | 83.49 | 2.41 | 0.0260 | 36.03 | 1.51 | n.s. |

T = t-test score;
p = probability;
n.s. = not significant;
Mean Diff. = mean of pre to post difference of 4 Hz
FMAER spectral power, μV²/Hz
For electrode locations see FIG. 1.

In FIG. 1, 4 Hz FMAER waveform data are shown within schematic ovals in vertex view with nose up, and left side of scalp to image left. The corresponding power spectra are shown to the immediate right. The top waveform and Fast Fourier Transform (FFT) displays were obtained prior to steroid administration. The bottom, corresponding displays were obtained after steroid administration. The vertical arrow to the lower left of each image represents 10 μV and the horizontal arrow beneath represents one second waveform length. The labels adjacent to the FMAER waveforms correspond to the standard EEG electrode 10-10 naming convention. Twenty-four electrodes' waveforms are shown. The FFT power spectral data horizontal axis covers the 0-30 Hz range. Note the near absent 4 Hz FMAER waveform response before and excellent 4 Hz waveform response after steroid administration. Note the spread of spectral power over many frequencies in the FFT display before (In FIG. 1A) which represents a distorted response. This contrasts to the nearly perfect 4 Hz response after steroid treatment (In FIG. 1B) which shows little spectral spread (little distortion).

In FIG. 1, for the vertex view display, waveforms are shown overlying their standard “10-10” locations. For the FFT graphs, channel order from top to bottom is: F3, F4, C3, C4, P3, P4, O1, O2, Fp1, Fp2, F7, F8, T7, T8, P7, P8, FT9, FT10, TP9, TP10, Fz, Cz, Pz, Oz. The common average reference is utilized for the displayed data (a reference free or “rfr” technique) [10].

In FIG. 1, abbreviations are used as follows: A=anterior, P=posterior, L=left, R=right, FMAER=4 Hz frequency modulated auditory evoked response, FFT=fast Fourier transform—power spectrum analysis shown as μV²/Hz, μV=microvolt, and Hz=Hertz or cycles per second.

FIG. 1 illustrates an example of one STAR group subject's relevant FMAER and associated spectral changes from before to after steroid administration: Note the absence of a clear 4 Hz sine wave following response before treatment in contrast to the excellent 4 Hz sine wave following response after treatment. The “before treatment” fast Fourier transform (FFT) power spectrum response furthermore illustrates that the input 4 Hz stimulus appears at the cortex and demonstrates power at many different frequencies aside from the expected 4 Hz input response. This occurrence of spurious response frequencies in output to a single frequency input represents response distortion [37]. It is visualized in the FMAER waveforms by their non-sinusoidal appearance and in the FFT by the spread across the spectrum away from the primary 4 Hz driving frequency. Note, however, that after steroids the FMAER waveform was sinusoidal at 4 Hz and the FFT showed a well-aligned response at the expected 4 Hz frequency without spectral spread, i.e., without distortion. All STAR subjects uniformly demonstrated such reduction of distortion after steroid treatment, while this change was not observed in the NSA group.

Noise Analysis: As summarized in Table 4B, for the first study point, as shown in Table 4B-(a), there was significantly increased Vrms in the standard average as compared to the plus-minus average at both electrodes selected (TP9 p≦0.0046, C3 p≦0.0012). Also at the first study point, both TP9 and C3 demonstrated significantly increased 4 Hz activity for the standard average as compared to the plus-minus average (TP9 p≦0.0030, C3 p≦0.0014). However, there was also spuriously increased 5 Hz activity (TP9 p≦0.0388, C3 p≦0.0083) and 6 Hz (C3 p≦0.0278). These findings indicate that at the first study point in addition to an unexpected 4 Hz response there was also evidence of a strong noise response at frequencies other than 4 Hz, namely at 5 Hz and 6 Hz. In contrast, at the second study point, as shown in Table 4B-(b), there was an even stronger 4 Hz response (TP9 p≦0.0017, C3 p≦0.0072), and moreover, this response now showed no evidence of significant accompanying noise responses at other frequencies. The 4 Hz FMAER response amplitude increase from time 1 to time 2, as shown in Table 4B-(c) remained statistically significant when corrected for noise at approximately the same statistical significance level as shown in Table 4A for the uncorrected response.

TABLE 4B
Noise analysis of the FMAER for Time 1 and Time 2, STAR group.
(a) Time One Pre-Treatment Data: Standard vs. Plus-Minus FMAER

| | | | | | | | | | 2, 3, |
|---|---|---|---|---|---|---|---|---|---|
| | Vrms | | 4 Hz | | 5 Hz | | 6 Hz | | 7 Hz |
| Electrode | T | p | T | p | T | p | T | p | p |
| C3 | +3.84 | 0.0012 | +3.77 | 0.0014 | +2.97 | 0.0083 | +2.39 | 0.0278 | n.s. |
| TP9 | +3.24 | 0.0046 | +3.43 | 0.0030 | +2.26 | 0.0388 | n.s. | | n.s. |

(b) Time Post-Treatment Data: Standard vs. Plus-Minus FMAER

| | 4 Hz | | 2, 3, 5, 6, 7 Hz |
|---|---|---|---|
| Electrode | T | p | p |
| C3 | +5.96 | 0.0000 | n.s. |
| TP9 | +5.94 | 0.0000 | n.s. |

(c) Time 1 vs. Time 2: Noise Corrected Spectral Data

| | 4 Hz | |
|---|---|---|
| Electrode | T | p |
| C3 | +3.03 | 0.0072 |
| TP | +3.69 | 0.0017 |

Change in EEG Abnormality between STAR and NSA Groups: As demonstrated in Table 5, there was no significant difference across time points in the type of EEG abnormalities identified by visual inspection between the STAR and NSA groups by Fisher's exact test. Thus, the children's EEG, in contrast to the FMAER, failed to show any effect associated with of time or treatment.

TABLE 5
Difference between groups in EEG change from Time 1 to Time 2
EEG CHANGE SUMMARY:

| | | No | | by Fisher 2 × 3 |
|---|---|---|---|---|
| Group | Worse | Change | Improved | exact test |
| STAR | 7 | 10 | 3 | n.s. |
| NSA | 4 | 12 | 8 | |

Change in Language Function As demonstrated in Table 6, the STAR group showed a significant difference in the mean CLSQ scores from time 1 to time 2; receptive and expressive language functions both showed a highly significant score increase, i.e., improvement. For the NSA group the mean of the language change score for receptive language was 0.167 and for expressive language 0.542; neither reached statistical significance.

TABLE 6
Time 1 to Time 2 CLSQ difference scores for STAR group

| | Mean | | |
|---|---|---|---|
| Language | Diff. | T | p |
| Receptive | 4.80 | 7.32 | 0.00001 |
| Expressive | 4.10 | 6.17 | 0.00001 |

t = t-test score;
p = probability;
n.s. = not significant;
Mean Diff. = mean of pre- to post difference of CLSQ difference scores

Number of Subjects Showing Change in Language Function: Negative language change scores indicated language worsening, a score of zero indicated lack of change, and positive scores indicated language improvement from the first to the second visit. Table 7 shows two 3×2 Fisher's exact tests for the number of subjects showing change (improvement, no change, or worsening) separately for receptive and expressive language. The results revealed improvement for a significantly higher number of STAR group subjects as compared to NSA group subjects, both in terms of receptive as well as expressive language.

TABLE 7
Time 1 to Time 2 Change in language scores between
first and second study for STAR and NSA groups

| | Group | Better | NoDiff | Worse | Fisher Exact |
|---|---|---|---|---|---|
| Receptive | STAR | 17 | 3 | 0 | P ≦ 0.0002 |
| Language | NSA | 6 | 16 | 2 | |
| Expressive | STAR | 17 | 2 | 1 | P ≦ 0.0031 |
| Language | NSA | 10 | 13 | 1 | |

p = probability;
NoDiff = no difference

Language Change for NSA Subjects With and Without History of Regression: Table 8 compares receptive and expressive language change scores across the two time points for the number of NSA group subjects, who had a history of regression with those who did not show regression. As 2×2 Fisher exact test subject numbers per cell were small, subjects who showed no change or worsened were collapsed into one group. The results revealed that the number of children without a history of regression did not statistically differ in terms of language change over time from the number of subjects who did not show regression.

TABLE 8
Time 1 to Time 2 Change in language scores for NSA group when
comparing the patients with and without history of regression

| | | | NoDiff/ | Fisher |
|---|---|---|---|---|
| | Group | Better | Worse | Exact |
| Receptive | NoRegr | 3 | 14 | n.s. |
| Language | Regr | 3 | 4 | |
| Expressive | NoRegr | 7 | 10 | n.s. |
| Language | Regr | 3 | 4 | |

Relationship Between FMAER and Language Change Scores for the STAR and NSA Groups Separately: Table 9 shows the STAR group's result of the stepwise multiple regression analysis performed in order to explore the relationship between changes in the FMAER and changes in language performance separately for receptive and expressive language across each subject's two time points. The result was highly significant with just a single FMAER variable chosen for receptive language (FMAER at C3, left central region) and a single variable chosen for expressive language (FMAER at T7, left mid-temporal region). The first step in both analyses showed many of the 14 independent variables' significant correlation with the dependent language measure (receptive 8/14, expressive 9/14 at F≧4.0 to enter). However, at the second step after the removal of the effect of the first independent variable from the remaining 13 independent variables, none of the variables reached the F level≧4.0; therefore none was chosen. This indicates suppression of 13 variables by the first variable chosen, which in turn demonstrates that, as expected, that all 14 independent variables contain similar information. Table 10 shows the analogous result for the NSA group. Again the result was highly significant with a single variable (FMAER at P4, right parietal lobe) chosen for both the receptive and the expressive language scores. Again there was only one step, since the first variable chosen suppressed the remaining 13 variables due to the high shared information, i.e. high correlation among the variables.

TABLE 9
Multiple regression, STAR group: 14 FMAER differential
FFT scores separately predict receptive and expressive
language differential scores

| Variable Entered | R | p | F to enter |
|---|---|---|---|
| Receptive | 0.6296 | 0.01 | 11.82 |
| C3 | | | |
| Expressive | 0.6134 | 0.01 | 10.86 |
| T7 | | | |

TABLE 10
Multiple regression, NSA group: 14 FMAER differential
FFT scores separately predict receptive and expressive
language differential scores

| Variable Entered | R | p | F to enter |
|---|---|---|---|
| Receptive | 0.5909 | 0.01 | 11.81 |
| P4 | | | |
| Expressive | 0.5288 | 0.01 | 8.54 |
| P4 | | | |

Thus, although similarly significant multiple FMAER-language regression scores were noted for both groups, the best FMAER-language correlation for the STAR group manifested itself in two left hemisphere electrodes (T7 receptive, C3 expressive) whereas for the NSA group the best language correlation manifested itself in a single right hemisphere electrode (P4 right parietal, for both, receptive and expressive).

Change in Behavioral Criteria for Autistic Disorder, STAR Group: As shown in Table 11, the STAR group showed a highly significant reduction in the DSM-IV ASD scaled symptom summary scores when comparing the before and after treatment scores.

TABLE 11
Time 1 to Time 2 DSM-IV Score difference, STAR Group

| | | | | |
|---|---|---|---|---|
| | Average | | | |
| | DSM-IV | | | |
| Group | Score | d.f. | T | p |

t-test of mean of 14 scores (see text and Table 2)

| | | | | |
|---|---|---|---|---|
| Before | 2.0677 | 38 | 7.261 | 0.00001 |
| After | 0.9857 | | | |

For the STAR group, 9 of 14 electrodes showed markedly increased second-study 4 Hz response magnitudes with maximal effect in the left inferior-posterior temporal electrode, TP9 (p≦0.00037). In contrast the NSA group failed to manifest any significant change in 4 Hz spectral power at any of the 14 electrodes Thus, the steroid treatment appears associated with a significant increase in the specific FMAER stimulation elicited 4 Hz response amplitude of the superior temporal gyri (STG) in both hemispheres of the study children with regressive autism. Additionally, the FMAER response distortion present at the first study point of the STAR group was absent at the second study point (Table 4B) Thus, the STAR group demonstrated both higher amplitudes and a less distortion in the FMAER after steroid treatment.

It is hypothesized that language regression in autism may result from the development of dysfunction in the specific STG systems that are needed to accurately and cleanly detect rapidly changing spectral information within the acoustic stream. Children with R-ASD are not deaf or “hard-of-hearing” per se; they often clearly identify even very soft, novel sounds. The problem appears to reside in the auditory distortion that occurs within cortex devoted to language processing. This distortion appears to be at least partly reversible with steroid therapy.

The study's second goal was to determine whether the significant FMAER improvements might be related to steroid-based suppression of presumed-to-be minor abnormalities in the children's EEGs as observed by visual inspection. Note that neither group demonstrated frank epileptiform transients such as spikes or spike and wave patterns. The EEG change rankings did not differ between the two groups (Table 5). Additionally, the EEG changes in the STAR group included both EEG improvement and EEG worsening. These findings emphasize that there appears to be little evidence for a physiologically meaningful steroid effect on EEG. This is an important finding that runs contrary to the assumption made in Landau-Kleffner syndrome, namely that both language and FMAER improvements with steroids result from suppression of frequent spike discharges within the STG [16]. What may be common to R-ASD and LKS children's brains is dysfunction of the STG. However, the dysfunction need not necessarily be accompanied by spike discharges. It may, none-the-less, be ameliorated by steroids.

The third goal was to compare changes in clinical-rating-based language scores between the first and the second study. As shown in Table 6, the STAR group showed highly significant improvement in the language ratings between time study 1 and 2; the improvement was comparable for both the receptive and expressive language ratings. Moreover, significantly more STAR group subjects (17/20) than NSA group subjects showed improvement (6/24 “better” receptive, and 10/24 “better” expressive). These data suggest that steroid treatment may be associated with improvement in language and that more subjects who receive steroid treatment may show such improvement than subjects in the non-treated group.

In order to rule out the possibility that regressive autism spontaneously improves with time, estimated language change of the seven NSA group children with histories of regression was compared to that of the 17 NSA group children without such a history of regression (Table 3). Although the small population size precludes a definitive answer, there was no significant difference between the two NSA subpopulations for receptive or for expressive language ratings (Table 8). Thus, spontaneous NSA group improvement of language in the regressive autism subpopulation was not observed.

The fourth goal was to explore the possible relationships between changes in 4 Hz spectral responses at all electrodes and the language ratings change scores (Table 6) for receptive and expressive language separately. The STAR group (Table 9) showed a strong correlation between receptive language and the FMAER at the left central region (C3), and between expressive language and the FMAER in the left mid temporal region (T7). It appears clinically meaningful that STAR group language improvement was more evident in the left hemisphere as the left hemisphere is typically the dominant hemisphere for language. Given that the scalp FMAER data are highly inter-correlated and reflect activity at the level of the STG, the difference in the scalp location of maximal effect for correlations with receptive and expressive language is curious. It is possible that slightly different regions of the STG are responsible for expressive and receptive language functions resulting in subtle differences in the dipole source orientations or locations and correspondingly different in patterns of scalp projection. This is an area for future exploration. For the NSA group (Table 10) a strong correlation was found between the right parietal (P4) region and both the receptive and the expressive language scores. It is curious that the typically non-language dominant right hemisphere STG maximally correlated with NSA group language function. This may reflect relatively greater dysfunction within the left hemisphere in this group. These findings are limited by the clinical rating measures of language employed in this study. Formal language testing with standardized assessments will be required to substantiate these preliminary findings.

The fifth goal was to determine the level of behavioral improvement within the steroid-treated group. The scaled DSM-IV symptom summary scores demonstrated a highly significant improvement after steroid treatment (p≦0.00001) (Table 11). Thus steroid treatment may be associated with behavioral improvement.

### CONCLUSIONS

Each of the above identified modules or programs correspond to a set of instructions for performing a function described above. These modules and programs (i.e., sets of instructions) need not be implemented as separate software programs, procedures or modules, and thus various subsets of these modules may be combined or otherwise re-arranged in various embodiments. In some embodiments, memory may store a subset of the modules and data structures identified above. Furthermore, memory may store additional modules and data structures not described above.

The illustrated aspects of the disclosure may also be practiced in distributed computing environments where certain tasks are performed by remote processing devices that are linked through a communications network. In a distributed computing environment, program modules can be located in both local and remote memory storage devices.

Moreover, it is to be appreciated that various components described herein can include electrical circuit(s) that can include components and circuitry elements of suitable value in order to implement the embodiments of the subject innovation(s). Furthermore, it can be appreciated that many of the various components can be implemented on one or more integrated circuit (IC) chips. For example, in one embodiment, a set of components can be implemented in a single IC chip. In other embodiments, one or more of respective components are fabricated or implemented on separate IC chips.

What has been described above includes examples of the embodiments of the present invention. It is, of course, not possible to describe every conceivable combination of components or methodologies for purposes of describing the claimed subject matter, but it is to be appreciated that many further combinations and permutations of the subject innovation are possible. Accordingly, the claimed subject matter is intended to embrace all such alterations, modifications, and variations that fall within the spirit and scope of the appended claims. Moreover, the above description of illustrated embodiments of the subject disclosure, including what is described in the Abstract, is not intended to be exhaustive or to limit the disclosed embodiments to the precise forms disclosed. While specific embodiments and examples are described herein for illustrative purposes, various modifications are possible that are considered within the scope of such embodiments and examples, as those skilled in the relevant art can recognize.

In particular and in regard to the various functions performed by the above described components, devices, circuits, systems and the like, the terms used to describe such components are intended to correspond, unless otherwise indicated, to any component which performs the specified function of the described component (e.g., a functional equivalent), even though not structurally equivalent to the disclosed structure, which performs the function in the herein illustrated exemplary aspects of the claimed subject matter. In this regard, it will also be recognized that the innovation includes a system as well as a computer-readable storage medium having computer-executable instructions for performing the acts and/or events of the various methods of the claimed subject matter.

The aforementioned systems/circuits/modules have been described with respect to interaction between several components/blocks. It can be appreciated that such systems/circuits and components/blocks can include those components or specified sub-components, some of the specified components or sub-components, and/or additional components, and according to various permutations and combinations of the foregoing. Sub-components can also be implemented as components communicatively coupled to other components rather than included within parent components (hierarchical). Additionally, it should be noted that one or more components may be combined into a single component providing aggregate functionality or divided into several separate sub-components, and any one or more middle layers, such as a management layer, may be provided to communicatively couple to such sub-components in order to provide integrated functionality. Any components described herein may also interact with one or more other components not specifically described herein but known by those of skill in the art.

In addition, while a particular feature of the subject innovation may have been disclosed with respect to only one of several implementations, such feature may be combined with one or more other features of the other implementations as may be desired and advantageous for any given or particular application. Furthermore, to the extent that the terms “includes,” “including,” “has,” “contains,” variants thereof, and other similar words are used in either the detailed description or the claims, these terms are intended to be inclusive in a manner similar to the term “comprising” as an open transition word without precluding any additional or other elements.

As used in this application, the terms “component,” “module,” “system,” or the like are generally intended to refer to a computer-related entity, either hardware (e.g., a circuit), a combination of hardware and software, software, or an entity related to an operational machine with one or more specific functionalities. For example, a component may be, but is not limited to being, a process running on a processor (e.g., digital signal processor), a processor, an object, an executable, a thread of execution, a program, and/or a computer. By way of illustration, both an application running on a controller and the controller can be a component. One or more components may reside within a process and/or thread of execution and a component may be localized on one computer and/or distributed between two or more computers. Further, a “device” can come in the form of specially designed hardware; generalized hardware made specialized by the execution of software thereon that enables the hardware to perform specific function; software stored on a computer-readable medium; or a combination thereof.

Moreover, the words “example” or “exemplary” are used herein to mean serving as an example, instance, or illustration. Any aspect or design described herein as “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

Computing devices typically include a variety of media, which can include computer-readable storage media and/or communications media, in which these two terms are used herein differently from one another as follows. Computer-readable storage media can be any available storage media that can be accessed by the computer, is typically of a non-transitory nature, and can include both volatile and nonvolatile media, removable and non-removable media. By way of example, and not limitation, computer-readable storage media can be implemented in connection with any method or technology for storage of information such as computer-readable instructions, program modules, structured data, or unstructured data. Computer-readable storage media can include, but are not limited to, RAM, ROM, EEPROM, flash memory or other memory technology, CD-ROM, digital versatile disk (DVD) or other optical disk storage, magnetic cassettes, magnetic tape, magnetic disk storage or other magnetic storage devices, or other tangible and/or non-transitory media which can be used to store desired information. Computer-readable storage media can be accessed by one or more local or remote computing devices, e.g., via access requests, queries or other data retrieval protocols, for a variety of operations with respect to the information stored by the medium.

On the other hand, communications media typically embody computer-readable instructions, data structures, program modules or other structured or unstructured data in a data signal that can be transitory such as a modulated data signal, e.g., a carrier wave or other transport mechanism, and includes any information delivery or transport media. The term “modulated data signal” or signals refers to a signal that has one or more of its characteristics set or changed in such a manner as to encode information in one or more signals. By way of example, and not limitation, communication media include wired media, such as a wired network or direct-wired connection, and wireless media such as acoustic, RF, infrared and other wireless media.

In view of the exemplary systems described above, methodologies that may be implemented in accordance with the described subject matter will be better appreciated with reference to the flowcharts of the various figures. For simplicity of explanation, the methodologies are depicted and described as a series of acts. However, acts in accordance with this disclosure can occur in various orders and/or concurrently, and with other acts not presented and described herein. Furthermore, not all illustrated acts may be required to implement the methodologies in accordance with the disclosed subject matter. In addition, those skilled in the art will understand and appreciate that the methodologies could alternatively be represented as a series of interrelated states via a state diagram or events. Additionally, it should be appreciated that the methodologies disclosed in this specification are capable of being stored on an article of manufacture to facilitate transporting and transferring such methodologies to computing devices. The term article of manufacture, as used herein, is intended to encompass a computer program accessible from any computer-readable device or storage media.

Although some of various drawings illustrate a number of logical stages in a particular order, stages which are not order dependent can be reordered and other stages can be combined or broken out. Alternative orderings and groupings, whether described above or not, can be appropriate or obvious to those of ordinary skill in the art of computer science. Moreover, it should be recognized that the stages could be implemented in hardware, firmware, software or any combination thereof.

The foregoing description, for purpose of explanation, has been described with reference to specific embodiments. However, the illustrative discussions above are not intended to be exhaustive or to be limiting to the precise forms disclosed. Many modifications and variations are possible in view of the above teachings. The embodiments were chosen and described in order to best explain the principles of the aspects and its practical applications, to thereby enable others skilled in the art to best utilize the aspects and various embodiments with various modifications as are suited to the particular use contemplated.

### REFERENCES

1. Baio J: Prevalence of Autism Spectrum Disorders—Autism and Developmental Disabilities Monitoring Network, 14 Sites, United States, 2008. In., vol. 61(SS03). Atlanta, Ga. National Center on Birth Defects and Developmental Disabilities, CDC; 2012: 1-19.
2. Barger B D, Campbell J M, McDonough J D: Prevalence and onset of regression within autism spectrum disorders: a meta-analytic review. J Autism Dev Disord, 43(4):817-828.
3. Rogers S J, Dilalla D L: Age of Symptom Onset in Young Children with Pervasive Developmental Disorders. J Am Acad Child Adol Psychiat 1990, 29(6):863-872.
4. Stefanatos G A: Regression in autistic spectrum disorders. Neuropsychol Rev 2008, 18(4):305-319.
5. Ozonoff S, Iosif A M, Baguio F, Cook I C, Hill M M, Hutman T, Rogers S J, Rozga A, Sangha S, Sigman M et al: A prospective study of the emergence of early behavioral signs of autism. J Am Acad Child Adolesc Psychiatr 2010, 49(3):256-266.e251-252.
6. Miller D T, Adam M P, Aradhya T, Biesecker L G, Brothman A R, Carter N P, Church D M, Crolla J A, Eichler E E, Epstein C J et al: Consensus Statement: Chromosomal Microarray Is a First-Tier Clinical Diagnostic Test for Individuals with Developmental Disabilities or Congenital Anomalies. Am J Human Gen 2010, 86(5):749-764.
7. Mantovani J F: Autistic regression and Landau-Kleffner syndrome: progress or confusion? Dev Med Child Neurol 2000, 42(5):349-353.
8. Stefanatos G A, Kinsbourne M, Wasserstein J: Acquired epileptiform aphasia: A dimensional view of Landau-Kleffner syndrome and the relation to regressive autistic spectrum disorders. Child Neuropsychol 2002, 8(3):195-228.
9. Fernandez I S, Peters J, Takeoka M, Rotenberg A, Prabhu S, Gregas M C, Riviello J M, Kothare S, Loddenkemper T: Patients with electrical status epilepticus in sleep share similar clinical features regardless of their focal or generalized sleep potentiation of epileptiform activity. J Child Neurol 2013, 28(1):83-89.
10. Lord C, Rutter M, DiLavore P C, Risi S, Gotham K: Autism Diagnostic Observation Schedule—Second Edition (ADOS-2). Torrence, Calif.: Western Psychological Services; 2012.
11. Le Couteur A, Lord C, Rutter M: The Autism Diagnostic Interview-Revised (ADI-R) In., edn.: Western Psychological Services; 2003.
12. Tsuru T, Mori M, Mizuguchi M, Momoi M Y: Effects of high-dose intravenous cortisteroid therapy in Landau-Kleffner syndrome. Pediat Neurol 2000, 22:145-147.
13. Sinclair D B, Snyder T J: Corticosteroids for the Landau-Kleffner syndrome and continuous spike-wave discharges during sleep. Pediat Neurol 2005, 32:300-306.
14. Lesca G, Rudolf G, Labalme A, Hirsch E, Arzimanoglou A, Genton P, Motte J, de Saint Martin A, Valenti M P, Boulay C et al: Epileptic encephalopathies of the Landau-Kleffner and continuous spike and waves during slow-wave sleep types: genomic dissection makes the link with autism. Epilepsia 2012, 53(9):1526-1538.
15. Lewine J D, Andrews R, Chez M, Patil A A, Devinsky O, Smith M, Kanner A, Davis J T, Funke M, Jones G et al: Magnetoencephalographic patterns of epileptiform activity in children with regressive autism spectrum disorders. Pediatrics 1999, 104(3 Pt 1):405-418.
16. Duffy F H, Eksioglu Y Z, Rotenberg A, Madsen J R, Shankardass A, Als H: The frequency modulated auditory evoked response (FMAER), a technical advance for study of childhood language disorders: cortical source localization and selected case studies. BMC Neurol 2013, 13(1):1-22.
17. Baram T Z: Pathophysiology of massive infantile spasms: Perspective on the putative role of the brain adrenal axis. Ann Neurol 1993, 33:231-236.
18. Kurian M, Korff C M: Steroids in pediatric epilepsy: infantile spasms and beyond. Epileptol 2011, 28:15-20.
19. Stafstrom C E, Arnason B G, Baram T Z, Catania A, Cortez M A, Glauser T A, Pranzatelli M R, Riikonen R, Rogawski M A, Shinnar S et al: Treatment of infantile spasms: emerging insights from clinical and basic science perspectives. J Child Neurol 2011, 26(11):1411-1421.
20. Connolly A M, Chez M G, Pestronk A, Arnold S T, Mehta S, Deuel R K: Serum autoantibodies to brain in Landau-Kleffner variant, autism, and other neurologic disorders. J Pediatr 1999, 134(5):607-613.
21. Martin L A, Ashwood P, Braunschweig D, Cabanlit M, Wan de Water J, Mmaral D G: Stereotypies and hyperactivity in rhesus monkeys exposed to IgG from mothers of children with autism. Brain Behav Immunity 2008, 22:806-816.
22. Chez M G, Guido-Estrada N: Immune therapy in autism: historical experience and future directions with immunomodulatory therapy. Neurotherapeutics 2010, 7(3):293-301.
23. Kovarik J: From immunosuppression to immunomodulation: current principles and future strategies. Pathobiol 2013, 80(6):275-281.
24. Shenoy S, Arnold S, Chatila T: Response to steroid therapy in autism secondary to autoimmune lymphoproliferative syndrome. J Pediatr 2000, 136(5):682-687.
25. Stefanatos G A, Grover W, Geller E: Case study: Corticosteroid treatment of language regression in pervasive developmental disorder. J Am Acad Child Adolesc Psychiat 1995, 34(8):1107-1111.
26. Landau W M, Kleffner F R: Syndrome of acquired aphasia with convulsive disorder in children. Neurol 1957, 7:523-530.
27. Holmes G, Riviello J: Treatment of childhood idiopathic language deterioration with valproate. Epilepsy Behav 2001, 2:272-276.
28. Guideline thirteen: guidelines for standard electrode position nomenclature. American Electroencephalographic Society. J Clin Neurophysiol 1994, 11(1):111-113.
29. Green G G R, Kay R H, Rees A: Responses evoked by frequency-modulated sounds recorded from the human scalp. J Physiol 1979, 296:21-22P.
30. Green G G R, Rees A, Stefanatos G A: A method for recording evoked responses to frequency modulated sounds in man. J Physiol 1980, 307:10p.
31. Stefanatos G A, Green G G R, Ratcliff G G: Neurophysiological evidence of auditory channel anomalies in developmental dysphasia. Arch Neurol 1989, 46(August):871-875.
32. Stefanatos G A, Foley C, Grover W, Doherty B: Steady-state auditory evoked responses to pulsed frequency modulations in children. Electroencephalogr Clin Neurophysiol 1997, 104:31-42.
33. Stefanatos G A: Speech perceived through a damaged temporal window: Lesons from word deafness and aphasia. Semin Speech Lang 2008, 29(3):239-252.
34. van Drongelen W: Signal Processing for Neuroscientists: An Introduction to the Analysis of Physiological Signals vol. 5. Oxford Elsevier; 2011.
35. Association AP: Diagnostic and Statistical Manual of Mental Disorders Fourth Edition Text Revision (DSM-IV-TR). Washington, D.C.: American Psychiatric Publishing, Inc.; 2000.
36. Dixon W J: BMDP Statistical Software (revised edition). Berkeley: University of California Press; 1985.
37. Wu S, Pols L: A distance measure for objective quality evaluation of speech communication channels using also dynamic spectral features. Proc Inst Phonetic Sci Amsterdam 1996, 20:27-42.
38. DeStefano F, Price C S, Weintraub E S: Increasing exposure to antibody-stimulating proteins and polysaccharides in vaccines is not associated with risk of autism. J Pediatr 2013, 162(2):561-567.
39. Chatham W W, Kimberly R P: Treatment of lupus with corticosteroids. Lupus 2001, 10(3):140-147.

1. A computer implemented method for detection of a biological condition, comprising:
on a device having one or more processors and a memory storing one or more programs for execution by the one or more processors, the one or more programs including instructions for:
measuring a first frequency modulated auditory evoked response of a subject with regressive autism spectrum disorder prior to administration of a therapeutic molecule to the subject;
measuring a second frequency modulated auditory evoked response of the subject after administration of the therapeutic molecule to the subject; and
analyzing a difference between the first and second frequency modulated auditory evoked response to detect the presence or absence of an improvement in at least one of language and behavior of the subject.

2. The method of claim 1, wherein the first and second frequency modulated auditory evoked responses are 4 Hz frequency modulated auditory evoked response steady state responses.

3. The method of claim 1, wherein the analyzing the difference step comprises spectral noise analysis.

4. The method of claim 1, wherein the analyzing the difference step comprises analyzing a difference between the average spectral power.

5. The method of claim 1, wherein the analyzing the difference step comprises determining a plus-minus average of the first and second frequency modulated auditory evoked response and a root-mean-square voltage of the plus-minus averages.

6. The method of claim 4, wherein the analyzing the difference step comprises analyzing a difference between the average spectral power of the 4 Hz frequency band.

7. The method of claim 4, wherein the analyzing the difference step comprises analyzing a difference between the average spectral power of frequencies adjacent to 4 Hz frequency band.

8. The method of claim 7, wherein the frequencies adjacent to the 4 Hz frequency band comprise the 5 Hz and 3 Hz spectral bands.

9. The method of claim 7, wherein a reduction in spectral power of the frequencies adjacent the 4 Hz frequent band under a threshold amount trigger a notification to a user interface indicating an improvement in a behavior or language of the subject.

10. The method of claim 1, wherein the analyzing step comprises a measurement of a 4 Hz spectral power and a 4 Hz signal to noise ratio taken during or after conclusion of an intervention.

11. The method of claim 1, further comprising instructions for providing quantitative, scaled measures of improvement of superior temporal gyri and receptive language function.

12. The method of claim 1, wherein the analyzing step comprises spectral analysis of a difference between standard and plus-minus signal averaging; and estimating stimulus generated noise content of an evoked response separate from a noise content from inadequate signal averaging.

13. The method of claim 1, wherein the detection of the presence or absence of an improvement in at least one of language and behavior of the subject, further comprises a quantitative indication in the improvement based on the difference.

14. The method of claim 13, wherein the quantitative indication comprises a numeric score.

15. The method of claim 14, wherein the quantitative indication is utilized to determine an appropriate dosage of a therapeutic molecule.

16. Then method of claim 1, wherein the therapeutic molecule is a corticosteroid.

17. A computer system for detection of a biological condition, comprising:
one or more processors; and
at least one memory to store one or more programs, the one or more programs comprising instructions for:
measuring a first frequency modulated auditory evoked response of a subject with autism spectrum disorder prior to administration of a therapy to the subject;
measuring a second frequency modulated auditory evoked response of the subject after administration of the therapy to the subject; and
analyzing a difference between the first and second frequency modulated auditory evoked response to detect the presence or absence of an improvement in language of the subject.

18. The computer system of claim 17, wherein the therapy is administration of a corticosteroid to the subject.

19. The computer system of claim 17, wherein the autism spectrum disorder is regressive autism spectrum disorder.

20. A non-transitory computer-readable storage medium storing one or more programs for detection of a biological condition, the one or more programs for execution by one or more processors of a computer system, the one or more programs comprising instructions for:
measuring a first frequency modulated auditory evoked response of a subject with autism spectrum disorder prior to administration of a therapy to the subject;
measuring a second frequency modulated auditory evoked response of the subject after administration of the therapy to the subject; and
analyzing a difference between the first and second frequency modulated auditory evoked response to determine the presence or absence of an improvement in language of the subject.

21. The non-transitory computer-readable storage medium of claim 20, where the therapy comprises administration of a corticosteroid to the subject.
